(19) United States
(12) Patent Application Publication
(10) Pub. No.: US 2011/0183353 A1
(43) Pub. Date: Jul. 28, 2011

(54) COMPOSITION AND METHOD FOR DIAGNOSING FUNGAL DISEASE

(76) Inventor: Daniel Poulain, Templeuve (FR)

(21) Appl. No.: 12/745,201

(22) PCT Filed: Dec. 1, 2008
(86) PCT No.: PCT/IB08/03929

§ 371 Date: Mar. 11, 2011

Related U.S. Application Data
(60) Provisional application No. 61/005,059, filed on Nov. 30, 2007.
(60) Provisional application No. 61/007,656, filed on Dec. 13, 2007.

(51) Int. Cl. G01N 33/569

(52) U.S. Cl. 435/7.1; 436/501

(57) ABSTRACT

Methods of diagnosing a fungal infection using anti-glycan antibodies alone or in combination with other anti-fungal diagnostic tests are described. Laminaribioside and chitobioside are used as antigens to detect human antibodies.

### RELATED APPLICATIONS SECTION

This application claims priority to U.S. Ser. No. 61/005,059, filed Nov. 30, 2007 and U.S. Ser. No. 61/007,656, filed Dec. 13, 2007, both of which are incorporated herein by reference in their entireties.

### FIELD OF THE INVENTION

The invention relates to diagnosing disease and more particularly to methods of diagnosing disease caused by fungi using anti-glycan antibodies alone or in combination with other diagnostic tests.

### BACKGROUND OF THE INVENTION

Over the past few decades Candida albicans has become one of the leading causes of nosocomial infection. Basic progress has been made in the understanding of C. albicans virulence attributes, the mechanisms of saprophytic-pathogenic transition, and factors predisposing patients to infection. However, despite this progress and increasing expenditure on antifungal therapy, both the incidence and attributable mortality of candidemia remain high (39-50%). This situation can be explained by the difficulty in establishing a reliable diagnosis of invasive Candida infection (ICI), particularly when blood cultures or tissue cultures, such as those from a liver biopsy or cerebrospinal fluid, are negative. As such, there is a need for a method that will allow sensitive and specific diagnosis of systemic fungal infection in at risk patients.

### SUMMARY OF THE INVENTION

The present invention is based, in part, upon the discovery that laminaribioside and chitobioside can be used as antigens to detect human antibodies that are specifically generated during early stage of fungal infections, e.g., an invasive fungal infection. An invasive or systemic fungal infection is an infection that occurs when fungal cells infect tissues, organs or other normally sterile sites, such as blood, liver, and cerebrospinal fluid. By contrast, non-invasive infections are typically characterized by the infection of non-sterile sites, such as the skin, mouth, vagina, and digestive tract.

These antigens are used for reliable diagnosis of invasive fungal infections even before a blood or tissue culture test indicates positive infection. Combined detection of anti-laminaribioside carbohydrate antibodies (ALCA) and anti-chitobioside carbohydrate antibodies (ACCA) provides earlier, and more accurate diagnosis of fungal infection than the previously reported anti-C. albicans mannans antibodies. Moreover, the detection tests of ACCA and ALCA antibodies are used to complement previously-described diagnostic tests for fungal infections, such as commercially available mannanemia detection tests.

The antigens are purified or synthetic. By “purified” or “substantially purified” is meant a laminaribioside or chitobioside molecule or biologically active portion thereof that is substantially free of cellular material or other contaminating macromolecules, e.g., polysaccharides, nucleic acids, or proteins, from the cell or tissue source from which the laminaribioside or chitobioside is derived. The phrase “substantially purified” also includes a laminaribioside or chitobioside molecule that is substantially free from chemical precursors or other chemicals when chemically synthesized. The language “substantially free of cellular material” includes preparations of laminaribioside or chitobioside that are separated from cellular components of the cells from which it is isolated.

Detection of ALCA complements the detection of circulating beta-1-3-glucans for diagnosis of invasive fungal infections. Detection of ACCA can also complement the detection of circulating chitin fragments from the wall debris of invasive fungal cells during infections. Considering that many major pathogenic fungal species include large quantities of glucans and chitin as essential components of their cell wall, ALCA and ACCA tests are also useful for diagnosing mycoses such as invasive aspergillosis, allergic bronchopulmonary aspergillosis, Pneumocystosis, either remotely, or in combination with other tests detecting circulating fungal specific molecules of these fungi, or the antibodies which bind to such molecules (e.g., detection of galactomannan and/or anti-galactomannan antibodies in sera of infected patients). Hyphomycetes, which are considered as emerging fungal pathogens, do not contain glucans in their cell wall; however, it contains large amounts of chitin, and therefore the ACCA test is useful for diagnosing tissue invasion by members of this fungal taxon.

Detection of ALCA and ACCA, individually or in combination with detection of anti-mannan from Saccharomyces cerevisiae antibodies (gASCA) assays, or in combination with other fungal diagnostic assays described herein, are useful for diagnosing fungal infections, including detecting systemic candidiasis before it can be diagnosed using blood culture. For example, a panel of assays for detection of gASCA, ALCA, ACCA, circulating C. albicans mannan (mannanemia), and antibodies to C. albicans mannan significantly improve diagnosis of fungal infections in comparison to traditional blood culture alone. This is particularly true for infections determined by non-C. albicans species for which the levels of mannan and anti-mannan antibodies could be low. For example, C. albican infection in rabbits was found to trigger anti-glycan antibody (ALCA, gASCA, and ACCA) responses.

Measuring circulating β1,3 glucans, in addition to ALCA, further improves the accuracy of the diagnosis, since patients with invasive fungemia are characterized as having either ALCA or circulating β1,3 glucans, detectable in sera. In addition to ACCA, measurement of circulating chitin fragments further improves the accuracy of the diagnosis, since patients with invasive fungemia are characterized as having either ACCA or circulating chitin fragments detectable in sera.

A wide range of diseases that are caused by a lack of tolerance to fungal antigens result in different types of hypersensitivity, ranging from immediate hypersensitivity (IgE mediated allergy), type III (immune complexes, mainly IgG) or type IV (delayed type hypersensitivity Chronic inflammation) hypersensitivity. Thus, the detection of ALCA and ACCA is also useful for diagnosis of fungal related allergic diseases, such as extrinsic allergic alveolitis (i.e. Farmer's lung and bronchopulmonary aspergillosis).

The invention provides methods of diagnosing a Candida albicans infection in a subject. First, a test blood sample is provided from a subject with symptoms of a Candida albicans infection. Next the level of an anti-GlcNAc (β1-4) GlcNAc (β) antibody (ACCA) and the level of an anti-Glc (β1-3) Glc (β) antibody (ALCA) in the test sample is detected by binding to a carbohydrate reagent comprising an isolated GlcNAc (β1-4) GlcNAc (β) glycan or an isolated Glc (β1-3) Glc (β) glycan, respectively. Candida albicans infection is diagnosed in the subject by detection of an elevated level of the antibodies in the test sample relative to a control sample from a control population of one or more individuals that do not have a Candida albicans infection or a reference level of the antibody. The reference level or range is determined by calculating the mean, median, or range level of each antibody in a population of one or more individuals that do not have a Candida albicans infection. Alternatively, the reference level is the level under which exists the antibody level of a majority of individuals that do not have an invasive Candida albicans infection, e.g., at least about 80-85%; at least about 85-90%; at least about 90-95%; or at least about 95-99% of individuals with antibody levels below the reference level do not have Candida albicans infection. The control population of individuals that do not have an invasive Candida albicans infection does not include individuals known to have Crohn's disease, ulcerative colitis, or celiac disease.

The invention also provides methods of diagnosing an invasive fungal infection in a subject by comparing amounts of ACCA or ALCA in a test sample from the subject to amounts of the antibody in a control sample from a control population of one or more individuals that do not have a fungal infection, or a control reference level. Optionally, the reference level is determined as the average amount/level of the antibody in the control sample population that do not have an invasive Candida albicans infection plus 2 to 3 standard deviations.

A fungal infection is diagnosed in the subject if the amount of at least one the antibody in the test sample is greater than the reference level or the amount of the antibody in the control sample, e.g., at least 5%, at least 10%, at least 25%, or at least 50% greater, or at least two fold, at least 5 fold, or at least 10 fold greater than a reference level or the amount of the antibody in the control sample.

In one aspect, the method comprises detecting ACCA and ALCA in the test sample, and diagnosing a fungal infection in the subject if the amount/level of the antibodies in the test sample are greater than the amount/level of the antibodies in the control sample or the reference level. In another aspect, the method further comprises detecting gASCA in the test sample, and diagnosing a fungal infection in the subject if the amount of the antibodies in the test sample are greater than the amount of the antibodies in the control sample or the reference level. In yet another aspect, the method further comprises detecting gASCA in the test sample, and diagnosing a fungal infection in the subject if the amount of the antibodies in the test sample is greater than the amount of the antibodies in the control sample or the reference level.

As used herein, the term “specificity” means the probability that a method is negative in the absence of the measured trait. Specificity is calculated as the number of true negative results divided by the sum of the true negatives and false positives. Specificity essentially is a measure of how well a method excludes those who do not have the measured trait. The anti-glycan cut-off value can be selected such that, when the sensitivity is at least about 60%, the specificity of diagnosing an individual is in the range of 80-85%, for example, 85-90%, 90-95%, or 95-99%.

Optionally, a fungal infection is diagnosed if the amount of the antibody is greater than the amount of the antibody in a control sample at a cutoff value providing specificity of at least about 80%. Alternatively, a fungal infection is diagnosed if the amount of the antibody is greater than the amount of the antibody in a control sample at a cutoff value providing specificity of at least about 85%. In another aspect, a fungal infection is diagnosed if the amount of the antibody is greater than the amount of the antibody in a control sample at a cutoff value providing specificity of at least about 90%. The invention also provides diagnosing a fungal infection if the amount of the antibody is greater than the amount of the antibody in a control sample at a cutoff value providing specificity of at least about 95%. The method further comprises comparing amounts of mannans in the test sample to a reference level. Optionally, the reference level is the average amount of mannans in a control sample from a control population of individuals that do not have an invasive fungal infection, plus 2 or 3 standard deviations. An invasive fungal infection is diagnosed if the manann amount in the test sample is higher then the amount of in the control population or reference level. In yet another aspect, the method further comprises comparing amounts of β(1,3) glucans in the test sample to the amounts of β(1,3) glucans in a control sample from a control population of one or more individuals that do not have a fungal infection or a reference level/range. A fungal infection is diagnosed if the β(1,3) glucans amount in the test sample is higher then the amount of β1,3 glucans in control population or reference level/range. The reference level or range is determined by calculating the mean, median, or range level of each antibody in a population of one or more individuals that do not have a Candida albicans infection. Alternatively, the reference level is the average amount of β(1,3) glucans in a control sample from a control population of individuals that do not have an invasive fungal infection, plus 2 to 3 standard deviations. An invasive fungal infection is diagnosed if the amount of β(1,3) glucans in the test sample is higher then the reference level.

Optionally, the method further comprises comparing the amount of chitin in the test sample to the amounts of chitin in a control sample from a control population of one or more individuals that do not have a fungal infection, wherein a fungal infection is diagnosed in the subject if the chitin amount in the test sample is higher then chitin amount in control population. Optionally, the reference level is the average amount of chitin in a control sample from a control population of individuals that do not have an invasive fungal infection, plus 2 to 3 standard deviations. An invasive fungal infection is diagnosed if the chitin amount in the test sample is higher then the reference level. The method further comprises comparing amounts of anti-mannan antibodies in the test sample to the amounts of anti-mannan antibodies in a control sample from a control population of one or more individuals that do not have a fungal infection. Optionally, the reference level is the average amount of anti-mannan antibodies in a control sample from a control population of individuals that do not have an invasive fungal infection, plus 2 or 3 standard deviations. An invasive fungal infection is diagnosed if the amount of anti-mannan antibodies in the test sample is higher then the reference level.

The diagnosis of fungal infection according to the described methods permits a reliable diagnosis even before conventional tests can detect fungal presence. In some situations, a sample is taken from the subject before the subject is positive in a blood culture test for the fungal infection. The methods are particularly useful in detection of a fungal infection caused by a Candida spp. An exemplary Candida spp. is Candida albicans. Alternatively, the fungal infection is systemic candidiasis, invasive aspergillosis, allergic bronchopulmonary aspergillosis, or pneumocystosis.

Levels of the antibodies are determined by direct or indirect detection. The amounts of the antibody are determined by measuring binding of the antibody to an isolated synthetic or purified glycan or a glycan attached via a linker to a solid phase, e.g., a substrate, a plate, or a chip. Alternatively, the amounts of the antibody are determined by measuring binding of the antibody to a polysaccharide containing the glycan antigen.

The test sample is a biological fluid. Exemplary biological fluids include whole blood, serum, plasma, spinal cord fluid, urine, tears and saliva. Preferably, the biological fluid is serum.

The invention also provides methods of diagnosing an invasive fungal infection in a subject by comparing amounts of ACCA in a test sample from the subject to amounts of the antibody in a control sample or reference level from a control population of one or more individuals that do not have a fungal infection. A fungal infection is diagnosed if the amount of the antibody in the test sample is greater than the amount of the antibody in the control sample, e.g., at least 5%, at least 10%, at least 25%, or at least 50% greater, or at least two fold, at least 5 fold, or at least 10 fold greater than a reference level or the amount of the antibody in the control sample. The invention also provides methods of diagnosing an invasive fungal infection in a subject by comparing amounts of ALCA in a test sample from the subject to amounts of the antibody in a control sample or reference level from a control population of one or more individuals that do not have a fungal infection and diagnosing a fungal infection in the subject if the amount of the antibody in the test sample is greater than the amount of the antibody in the control sample or reference level. Optionally, diagnosing an invasive fungal infection in a subject is carried out by measuring the amount/level of both ALCA and ACCA.

Also provided by the invention is a system for diagnosing a fungal infection in a subject. The system includes at least one memory operable to store data for amounts in a sample from the subject of one or more of an ACCA, ALCA and/or gASCA antibody. The system may optionally include at least one memory operable to store data for amounts of antibodies to C. albicans, amounts of mannans and/or amounts of circulating β1,3 glucans. The system also includes one or more processors, collectively operable to compare levels of the antibody to levels of the antibody to the level of the antibody in a control sample obtained from a subject known to not have fungal disease and to determine that the subject has fungal disease if higher amounts of the antibody are detected in the test sample as compared to the levels of the antibodies in the control sample.

The reagents and methods described herein overcome drawbacks associated with standard diagnostic methods for fungal infections such as Candidiasis, particularly systemic Candida infection (SCI). Common methods for diagnosing systemic or invasive Candidiasis include detecting Candida antigen by latex agglutination or antibody titer by indirect hemagglutination. Nevertheless, reliable diagnosis remains a major difficulty. Drawbacks associated with existing diagnostic methods include the following limitations. Clinical presentation is not specific, e.g., symptoms may mimic bacterial sepsis. Direct detection by culture, metabolites, or antigens has a low sensitivity.

ICU patients represent seriously sick patients with increased susceptibility to Candida infections. ICU patients are at risk for SCI because of predisposing factors such as intravenous lines, potent antibacterial treatment, as well as complicated histories such as alcoholism and diabetes. Standard, i.e., blood culture or histopathology, is not a reliable procedure, because of its very low sensitivity.

Systemic fungal infections include those involving infection of the bloodstream, and invasive infections include those which involve organs such as liver, lung, and kidney. Precise, reliable detection of fungal infection (i.e., before positive identification in a blood culture) is important to identify the subject as a candidate for antifungal therapy in addition or in place of administration of standard antibacterial agents.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Additional advantages of the methods described herein include accuracy and speed. For example, the test provides an answer in under 3 hours, e.g., under 2 hours, under 1 hour, under 30 minutes, or under 15 minutes. The clinician may therefore have an answer and determine therapy quickly. In contrast, blood culture requires several days and up to a week. A quick and definitive diagnosis is especially important with seriously ill patients, e.g., those in ICU, with complicated and life threatening conditions.

The invention will be illustrated in the following non-limiting examples. Other features and advantages of the invention will be apparent from the following detailed description and claims.

### BRIEF DESCRIPTION OF THE DRAWINGS

FIGS. 1A-1C are graphs showing the development of ALCA (FIG. 1A), gASCA (FIG. 1B), and ACCA (FIG. 1C) in New Zealand white rabbits following inoculated intravenously with suspensions of live cells of Candida albicans strain VW32.

FIG. 2 is a series of dot plots illustrating the distribution of ASCA, ALCA and ACCA in healthy controls (HC), patients with Crohn's disease (CD), ICU-patients with one or two body sites colonized by Candida species, but not having systemic or invasive Candida infection (CZ), and patients with invasive candidiasis (ICI). Comparison between each group of patients was performed using the Mann-Whitney U-test (P). For ASCA, a highly significant difference was observed for HC vs. CD (p<0.0001) and HC vs. ICI, while the difference between CD vs. ICI was not statistically significant. For ALCA, a highly significant difference was observed for HC vs. CD (p<0.0001) and HC vs. ICI (p<0.0001), while no difference was observed for CD vs. ICI. For ACCA, similar results were observed for HC vs. CD (p=0.001) and HC vs. ICI (p<0.0001). In contrast to ASCA and ALCA, a significant difference in ACCA was observed between ICI and CD patients (p=0.05). The same trend was observed for CZ, and no difference was observed for HC vs. CZ. The chemical structures of the antigens are presented on the right side of the figure; for ALCA and ACCA synthetic oligosaccharides are coated on the ELISA plates; ASCA* antigen is a natural antigen which comprises a repertoire of oligomannose epitopes, among these, the major epitopes supporting the humoral response in CD have been represented, since this synthetic analog was shown to specifically adsorb antibodies generated during C. albicans infection.

FIG. 3 is a series of bar graphs demonstrating the development of anti-C. albicans mannan antibodies (hatched bars), ASCA (black bars), ALCA (grey bars) and ACCA (white bars) in New Zealand white rabbits following intravenous inoculation of live C. albicans strain VW32 (a). Results are expressed as mean ODs±SE. Murine monoclonal antibody (mAb) 2G8 (black bars) and mAb EB-CA1 (grey bars) reactivities were determined by ELISA with laminaribioside, a synthetic analog of β-1,3 glucan involved in the ALCA test (b).

FIG. 4 is a panel of dot plots and bar graphs showing the results of screening sixty-nine serum samples from 18 patients with invasive Candida infection (ICI) for the presence of: (a) ASCA, (b) ALCA, (c) ACCA and (d) Platelia Ab (Plat.Ab) as described in the Materials and Methods. The antibody levels are plotted according to the date of serum sampling (day 0 indicates the date of mycological isolation of C. albicans from blood). The horizontal line indicates the cut-off values used to define positive and negative results. The vertical line indicates day 0. Antibody values (mean titers±SE) for each test are also presented as histograms (FIGS. a2, b2, c2 and d2) by classifying sera into four groups: Group 1 for sera taken during the period day-25 to day-1; group 2 (day 0 to day+15); group 3 (day+16 to day+40); and group 4 (day+41 to day+154).

FIG. 5 is a series of line graphs illustrating Examples of kinetic evolution of ASCA, ALCA, ACCA and Platelia Candida Ag and Ab tests in patients with proven invasive candidiasis. For both patients 14 (a) and 6 (b), a gradual decrease in ASCA, ALCA and ACCA was observed during the period preceding positive blood cultures to reach a minimum on day 0. After the candidemic episode, an overall increase was observed for most antibody markers during the proceeding weeks. For each Y axis, symbols indicate the cut-off values for serological tests.

### DETAILED DESCRIPTION OF THE INVENTION

Nosocomial fungal infections are a major public health problem affecting both immunocompromised and hospitalized patients (e.g., patients with hematologic and solid tumors; patients with transplants; and patients hospitalized for surgery or resuscitation). These infections are caused by opportunistic fungi of two types (yeasts and molds), which are commonly present in the digestive tract, on the skin, or in the patient's environment.

Particularly susceptible to fungal infections are those patients in intensive care units (ICU). The criteria for admission to ICU are well known, e.g., one of more of the following conditions: threatened airway, respiratory arrest, respiratory rate >40 or <8 breaths/min.; oxygen saturation <90% on >50% oxygen; cardiac arrest; pulse rate <40 or >140 beats/min.; systolic blood pressure <90 mm Hg; sudden fall in level of consciousness; repeated or prolonged seizures; and rising arterial carbon dioxide tension with respiratory acidosis.

The reagents and methods described herein are useful to reliably detect Candidiasis of those suffering from or at an risk of developing SCI in patients admitted to ICU or other critical care units. Fungal infection is detected even before a blood culture indicates positive identification of the fungus. Not only do the methods reliably detect systemic Candida infection (SCI), e.g., presence of fungal pathogens in the bloodstream, presence of the antibodies also reveals cases in which a potentially dangerous fungal infection is sequestered, e.g., in an organ such as liver or kidney. In the latter case, other methods such as blood culture would not reveal the infection, which could then proceed to SCI and become life threatening.

### Fungal Pathogens

Although numerous species of yeast and mold cause infections, the most pathogenic opportunistic fungal species are the yeast Candida albicans and the mold Aspergillus fumigatus. Factors that affect the type of fungal infection include the patient's underlying disease, and the medical and/or the surgical treatment the patient receives. Infections due to both Candida albicans and Aspergillus fumigatus are among the most prevalent hospital-acquired infections. Yeasts of the genus Candida rank fourth in hospital-acquired infections and are responsible for the highest attributable mortality (30-40%), while the mortality associated with invasive aspergillosis is estimated to be 60-80%. Many clinicians that are aware of the high risks incurred by their patients prescribe antifungal agents. The cost of these treatments reaches an alarming amount in several hospitals where it exceeds that of the antibacterial antibiotics. Despite the high cost of treatment, only a minimal decrease in morbidity and mortality is achieved with current antifungal agents.

The management of fungal infections is hampered by delays in diagnosis and the lack of reliable diagnostic methods which allow detection of fungemia, thus making therapeutic choice more difficult. Clinical signs of fungal infection are difficult to differentiate from bacterial infection, and mycological methods are insufficiently sensitive. The current “gold standard” for diagnosis of fungal infection is isolation of fungi from a normally sterile site in the body (e.g., blood, pericardial cerebrospinal fluids, biopsy specimens from liver, etc.) using culture. However, blood cultures are positive only in 50% of autopsy-confirmed cases of systemic candidiasis and biopsies are not practicable in a large majority of patients. In aspergillosis, the isolation of A. fumigatus from blood or biopsies is even more difficult to detect successfully. Although effective anti-fungal drugs do exist (e.g., amphotericin B, its lipid or liposomal formulations, and 5-fluorocytosine), the difficulty in diagnosis of fungal infections leads to either unnecessary treatment of patients or the lack of treatment when it is warranted.

As the opportunistic fungi are part of the normal human flora, the interpretation of values provided by diagnostic tests has to allow discrimination between the presence of fungi and the establishment of its pathogenicity based on quantitative or qualitative criteria. Numerous studies were designed to improve biological diagnosis by detecting circulating fungal molecules (proteins, glycans distributing in mannans and glucans, nucleic acids). However, prior to the invention described herein, detection of circulating fungal molecules lacked sensitivity and therefore, in many cases, patients test positive only at the later stages when infection is already established and more difficult to control. The methods described herein achieve sensitive and specific diagnosis of systemic fungal infection in patients under risk at the earliest stage of infection.

### Carbohydrates and Fungal Cell Structure

Not all species of fungi have cell walls but in those that do, the cell wall consists largely of chitin and other polysaccharides. The plasma membrane is followed by three layers of cell wall material, including (from the inside→out), (1) a chitin layer (polymer consisting mainly of unbranched chains of N-acetyl-D-glucosamine; (2) a layer of β-1,3-glucan; and (3) a layer of mannoproteins (mannose-containing glycoproteins) which are heavily glycosylated at the outside of the cell.

Pathophysiologically, mannans, glucans and chitin may be synthesized individually by various microbes which have adapted to the human gut. However, yeasts are the only organisms known to synthesize large quantities of each of these glycans in a single envelope. The present demonstration that ASCA, ALCA and ACAA are induced by C. albicans reinforces the serological observations indicating a link between this yeast and immune alterations observed in CD. In the case of fungal cell wall carbohydrates, the ubiquitous distribution of chitin and glucans indicates that ALCA and ACCA tests are useful in the diagnosis of other invasive mycoses.

### Candida sp.

The yeast cell wall consists of 80% glycans, 15% proteins and 5% lipids (Ruiz-Herrera J M et al., 2006 FEMS Yeast Res, 6:14-29). Glycans are distributed into 40% glucans (polymers of β-1,3 and β-1,6 glucose), 2-4% chitin, and 30% mannans. Mannans exist as mannoconjugates linked to proteins or lipids (Mille C et al., 2008 J Biol Chem, 283:9724-36). Human sera contain anti-C. albicans mannan antibodies whose synthesis has been suggested to be due to the natural presence of C. albicans in the gut (Kozel T R, et al., 2004 Infect Immun, 72:209-18). In hospitalized patients, an increase in C. albicans colonization (Samonis G A, et al., 1993 Antimicrob Agents Chemother, 37:51-3) slowly augments anti-mannan antibody levels whereas a sharp increase is generally associated with tissue invasion (Sendid B J et al., 2002 J Med Microbiol, 51:433-42). On this basis, regular survey of anti-mannan antibody levels in at-risk patients has been proposed as a strategy to compensate for the poor sensitivity of blood cultures. Depending on the method used, different cut-offs have been proposed to differentiate between colonized and infected patients (Prella M J et al., 2005 Diagn Microbiol Infect Dis, 51:95-101; Sendid B M et al., 1999 J Clin Microbiol, 37:15 10-7). In infected patients, a balance was observed between anti-mannan antibodies and mannanemia, and simultaneous screening for both markers has been recommended (Sendid 1999).

An important observation from this study is that C. albicans infection generates antibodies that can be detected with chitin oligomers. The presence of human antibodies against chitin was only investigated when synthetic chitobioside was discovered to be a biomarker for CD (Dotan I S et al., 2006 Gastroenterology 131:366-78). ACCA react with a minimal epitope composed of two units from a linear polymer of β-1,4-D-GlcNAc from chitin. Chitin is a component of the exoskeleton of arthropods, worm cuticles, protozoan cysts, and the cell wall of some algae, yeasts and filamentous fungi. Due to the abundance of organisms in the human environment and food, the presence of anti-chitin antibodies is not surprising even if the process of antibody generation is unknown.

Most invasive Candida infections (ICIs) are endogenous in origin, as revealed by genetic identity between strains isolated from the gut and blood cultures, as well the link between gut colonization and invasive infection (Piarroux R et al., 2004 Crit Care Med, 32:2443-9; Pittet D et al., 1994 Ann Surg, 220:751-8, Voss A, et al., 1994 J Clin Microbiol, 32:975-80). Despite this link, however, little research has focused on C. albicans in its natural niche (De Luca A et al., 2007 J Immunol, 179:5999-6008). Crohn's disease (CD) is an interesting topic for transversal research since this chronic inflammatory bowel disease is generally agreed to be triggered by genetic susceptibility to gut microbiota (Eckburg P B and Relman D A, 2007 Clin Infect Dis, 44:256-62). As the development of CD has also been linked to the sequential appearance of antibodies against microbial antigens (Ferrante M et al., 2007 Gut, 56:1394-403; Mow W S et al., 2004 Gastroenterology, 126:414-24.), it was investigated whether anti-C. albicans antibodies were associated with this disease. ASCA (anti-Saccharomyces cerevisiae antibodies) are widely used as serological markers of CD (Vernier G et al., 2004 Curr Gastroenterol Rep, 6:482-7). By using antibodies immunopurified on synthetic oligomannoses mimicking the major epitope of S. cerevisiae mannan supporting the ASCA response, it was demonstrated that this epitope is over-expressed by the pathogenic phase of C. albicans. Subsequently, it was shown that ASCA are serological markers of C. albicans infections in humans and animals (Jawhara S et al., 2008 J Infect Dis, 197:972-80; Standaert-Vitse A et al., 2006 Gastroenterology, 130:1764-75).

Screening of sera from patients with CD with a glycan array led to the identification of two new anti-glycan antibodies as serological markers of this disease (Dotan I, 2006 Gastroenterology, 131:366-78). Both antibodies are directed against molecular fragments corresponding to a laminaribioside (β1,3-linked glucose dimer) and chitobioside (β1,4-linked N-acetyl-glucosamine dimer). These antibodies have been labeled ALCA and ACCA, respectively, and complement the detection of ASCA as serological markers of CD. The combined detection of ASCA, ALCA and ACCA was named IBDX™, an acronym referring to Inflammatory Bowel Diseases. The cumulative presence of these antibodies corresponded to complicated disease with a higher risk of surgery (Ferrante M. L et al., 2007 Gut, 56:1394-403).

In addition to ASCA, ALCA and ACCA is also induced by C. albicans. Oligomers of β-D-1,3 glucose and β-1,4 linked N-acetyl-glucosamine are constitutive units of glucan and chitin, which are essential components of the yeast cell wall (Klis F M et al., FEMS Microbiol Rev, 26:239-56). The availability of the IBDX™ panel prompted the investigation of whether ALCA and ACCA, together with ASCA, could also be synthesized as a result of C. albicans infection. Prior to the invention described herein, the presence of anti-glucan and anti-chitin antibodies in patients infected by C. albicans had never been investigated since these cell wall components were previously considered to be non-immunogenic.

Fungal infections are detected according to methods of the invention using the anti-glycan antibodies ALCA and ACCA either individually, or in combination. Antibodies to these glycans may optionally be used along with antibodies to gASCA to diagnose fungal disease, as well as antibodies to C. albicans mannan. If desired, circulating mannans, β1,3 glucans, or chitin, are also detected, which further improves the accuracy of diagnosing fungal infections. Anti-glycan antibodies, circulating Mannans, circulating β1,3 glucans, circulating chitin, and, anti-C. albicans antibodies are detected using methods known in the art.

### Diagnostic Methods

Anti-glycan antibodies are typically detected using reagents that specifically bind to the anti-glycan antibodies. The reagents are, e.g., the specific glycan structures laminaribioside and chitobioside for ALCA and ACCA respectively. Alternatively, the reagents are other molecules or macromolecules or a polysaccharide that include the specific glycan structure.

The reagents that are used to specifically bind and detect those anti glycans antibodies are the specific glycan structures. Alternatively, the reagents are other molecules that include the specific glycan structure. The glycan or sugar structure is a purified carbohydrate moiety (including a monosaccharide, an oligosaccharide, or a polysaccharide) or such a carbohydrate is displayed on any solid phase, or a macromolecule or any other molecular structure that includes the glycan. The glycan-containing structure can be naturally occurring, e.g., extracted from an organism, or synthetic.

If desired, peptides that mimic carbohydrate antigens are used in the methods and compositions described herein. The peptides can be used to identify specific anti-glycan antibodies. Peptides which mimic structures recognized by anti-glycan antibodies can be identified using methods known in the art, e.g., by screening a filamentous phage-displayed random peptide library (Zhan et al., Biochem Biophys Res Commun, 308:19-22, 2003; Hou et al., J Immunol, 17:4373-79, 2003). ACCA is measured using the glycan fragment or the full polysaccharide. Thus, ACCA can be measured using the polysaccharide chitin as antigen, and ALCA can be measured using the polysaccharide laminarin, a glucose based polysaccharide with (Glc(beta 1,3)Glc(b) and Glc (beta 1,6)Glc(beta) connectivity, as antigen.

Glycan antigens used to identify various anti-glycan antibodies are obtained from a variety of other sources so long as the antigen is capable of binding specifically to the given anti-glycan.

### Detection of Glycans

Circulating Mannans, circulating beta 1,3 glucans, and circulating chitin, are detected using methods known in the art. The detection can be done, for example, by immuno-assay using a monoclonal of polyclonal antibody preparation that binds specifically to the polysaccharide (mannan, glucan, or chitin).

Detection of mannan is done, for example, by a commercially available ELISA test (Platelia Candida Ag test by Bio-Rad). This one step, sandwich, microplate EIA uses the MAb EB-CA1 as a captor and detector antibody to allow the detection of mannan in serum samples as described in (Boualem Sendid et al., 2004 Journal Of Clinical Microbiology, 164-171).

Detection of beta 1,3 glucan is accomplished, for example, by a commercially available test (Fungitell; associates of cape code Inc.) as described in Luis Ostrosky-Zeichner et al., 2005 Clinical Infectious Diseases, 41:654-659. Detection of chitin can be done for example as described by in the patent U.S. Pat. No. 5,004,699.

Binding to anti-glycan antibodies is performed using variety of other immunoassay formats known in the art, including competitive and non-competitive immunoassay formats can also be used (Self and Cook, 1996 Curr. Opin. Biotechnol. 7:60-65, which is incorporated by reference). Other assays include immunoassays, such as enzyme-linked immunosorbent assays (ELISAs). An enzyme such as horseradish peroxidase (HRP), alkaline phosphatase (AP), β-galactosidase or urease can be linked to a secondary antibody selective for a primary anti-glycan antibody of interest. A horseradish-peroxidase detection system can be used, for example, with the chromogenic substrate tetramethylbenzidine (TMB), which yields a soluble product in the presence of hydrogen peroxide that is detectable at 450 nm. An alkaline phosphatase detection system can be used with the chromogenic substrate p-nitrophenyl phosphate, for example, which yields a soluble product readily detectable at 405 nm. Similarly, a β-galactosidase detection system can be used with the chromogenic substrate o-nitrophenyl-a β-D-galactopyranoside (ONPG), which yields a soluble product detectable at 410 nm, or a urease detection system can be used with a substrate such as urea-bromocresol purple (Sigma Immunochemicals, St. Louis, Mo.). A useful secondary antibody linked to an enzyme can be obtained from a number of commercial sources; goat F(ab′)₂ anti-human IgG-alkaline phosphatase, for example, can be purchased from Jackson Immuno-Research (West Grove, Pa.).

Immunoassays encompass capillary electrophoresis based immunoassays (CEIA) and can be automated, if desired. Immunoassays also can be used in conjunction with laser induced fluorescence (See, for example, Schmalzing and Nashabeh, Electrophoresis 18:2184-93 (1997)); Bao, J. Chromatogr. B. Biomed. Sci. 699:463-80 (1997), each of which is incorporated herein by reference). Liposome immunoassays, such as flow-injection liposome immunoassays and liposome immunosensors, also can be used (Rongen et al., J. Immunol. Methods 204:105-133 (1997)).

A radioimmunoassay can also be used for determining whether a sample is positive for a glycan antibody, or for determining the level of anti-glycan antibodies in a sample. A radioimmunoassay using, for example, an ¹²⁵Iodine-labeled secondary antibody (Harlow and Lane, Antibodies A Laboratory Manual Cold Spring Harbor Laboratory: New York, 1988, which is incorporated herein by reference) is encompassed within the invention.

A secondary antibody may alternatively be labeled with a chemiluminescent marker. Such a chemiluminescent secondary antibody is convenient for sensitive, non-radioactive detection of anti-glycan antibodies and can be obtained commercially from various sources such as Amersham Lifesciences, Inc. (Arlington Heights, Ill.).

A detectable reagent may also be labeled with a fluorochrome. Appropriate fluorochromes include, for example, DAPI, fluorescein, Hoechst 33258, R-phycocyanin, B-phycoerythrin, R-phycoerythrin, rhodamine, Texas red or lissamine. A particularly useful fluorochrome is fluorescein or rhodamine. Secondary antibodies linked to fluorochromes can be obtained commercially. For example, goat F(ab′)₂ anti-human IgG-FITC is available from Tago Immunologicals (Burlingame, Calif.).

A signal from the detectable reagent can be analyzed, for example, using a spectrophotometer to detect color from a chromogenic substrate; a radiation counter to detect radiation, such as a gamma counter for detection of ¹²⁵Iodine; or a fluorometer to detect fluorescence in the presence of light of a certain wavelength. For detection of enzyme-linked reagents, a quantitative analysis of the amount of anti-glycan antibodies can be made using a spectrophotometer such as an EMAX Microplate Reader (Molecular Devices, Menlo Park, Calif.) in accordance with the manufacturer's instructions. If desired, the assays of the invention can be automated or performed robotically, and the signal from multiple samples can be detected simultaneously.

Other methods include, e.g., flow cytometry (including bead based immunoassays), and phage display technology for expressing a recombinant antigen specific for an anti-glycan antibody. Phage particles expressing the antigen specific for a desired anti-glycan antibody can be anchored, if desired, to a multiwell plate using an antibody such as an anti phage monoclonal antibody (Felici et al., “Phage-Displayed Peptides as Tools for Characterization of Human Sera” in Abelson (Ed.), Methods in Enzymol. 267, San Diego: Academic Press, Inc. (1996), which is incorporated by reference herein).

### Interpreting Binding Data

The amounts of anti-glycan antibodies (ALCA or ACCA or gASCA), mannans, β1,3 glucans, chitin, and/or anti-mannan antibodies in a test sample from a subject suspected of having an invasive or systemic fungal infection is compared to the amounts of anti-glycan antibodies, mannans, β(1,3) glucans, chitin, and/or anti-mannan antibodies in a control sample from a control population of one or more individuals that do not have a fungal infection. Optionally, the reference level is the level under which exists the antibody level of a majority of individuals that do not have an invasive Candida albicans infection, e.g., at least about 80-85%; at least about 85-90%; at least about 90-95%; or at least about 95-99% of individuals with antibody levels below the reference level do not have Candida albicans infection. The population of individuals that do not have an invasive Candida albicans infection does not include individuals known to have Crohn's disease, ulcerative colitis, or celiac disease. If the amount of anti-glycan antibodies, mannans, β(1,3) glucans, chitin, and/or anti-mannan antibodies in a test sample from a subject suspected of having a fungal disease is higher then a certain threshold reference level, the subject is suffering from an invasive or systemic fungal infection. In another aspect, the subject has not developed a systemic or invasive fungal infection. This threshold can be determined, for example, based on a the mean amount of anti-glycan antibodies, mannans, β(1,3) glucans, chitin, and/or anti-mannan antibodies in a control group not having invasive fungal infection, plus 2 or 3 standard deviations.

### Diagnostic Parameters

The clinical parameters of sensitivity, specificity, negative predictive value, positive predictive value and efficiency are calculated using true positives, false positives, false negatives and true negatives. A “true positive” sample is a sample positive for a fungal infection according to art-recognized methods for diagnosing the fungal infection, which is also diagnosed positive according to a method of the invention. A “false positive” sample is a sample negative by an art-recognized method, which is diagnosed positive according to a method of the invention. Similarly, a “false negative” is a sample positive for an art-recognized analysis, which is diagnosed negative according to a method of the invention. A “true negative” is a sample negative for the assessed trait by an art-recognized method, and also negative according to a method of the invention. See, for example, Mousy (Ed.), Intuitive Biostatistics New York: Oxford University Press (1995), which is incorporated herein by reference.

As used herein, the term “sensitivity” means the probability that a laboratory method is positive in the presence of the measured trait. Sensitivity is calculated as the number of true positive results divided by the sum of the true positives and false negatives. Sensitivity essentially is a measure of how well a method correctly identifies those with disease. In a method of the invention, the anti-glycan antibody values can be selected such that the sensitivity of diagnosing an individual is at least about 60%, and can be, for example, at least about 65%, 70%, 75%, 80%, 85%, 90% or 95%.

As used herein, the term “specificity” means the probability that a method is negative in the absence of the measured trait. Specificity is calculated as the number of true negative results divided by the sum of the true negatives and false positives. Specificity essentially is a measure of how well a method excludes those who do not have the measured trait. The anti-glycan cut-off value can be selected such that, when the sensitivity is at least about 60%, the specificity of diagnosing an individual is in the range of 80-85%, for example, 85-90%, 90-95%, or 95-99%.

The term “positive predictive value,” as used herein, is synonymous with “PPV” and means the probability that an individual diagnosed as having the measured trait actually has the disease. Positive predictive value can be calculated as the number of true positives divided by the sum of the true positives and false positives. Positive predictive value is determined by the characteristics of the diagnostic method as well as the prevalence of the disease in the population analyzed. In a method of the invention, the anti-glycan antibody cut-off values can be selected such that the positive predictive value of the method in a population having a disease prevalence of 15% is at least about 5%, and can be, for example, at least about 8%, 10%, 15%, 20%, 25%, 30% or 40%.

### EXAMPLE 1

### C. Albicans Infections In Rabbits Induces An Anti-Glycan Antibody Response

The relationship between C. albicans infections and the formation of ALCA, ACCA and gASCA was demonstrated as follows. Three New Zealand white rabbits (2-3 kg) were inoculated intravenously with 500 μL of suspensions of live yeasts of C. albicans VW32 2.106 yeasts/mL. Serum samples were drawn every week for 3 weeks and stored at −20° C. Serum antibodies against S. cerevisiae mannan (IgG ASCA), laminaribioside (IgG ALCA) and chitobioside (IgA ACCA) were detected by using ELISA tests (Glycominds, Lod, Israel) that were initially designed to detect human antibodies. Briefly, mannan, p-nitrophenyl laminaribioside and chitobioside were covalently bound to the surface of the microtiter plate wells with a linker (an oligomer of 1,8-diamino-3,6-dioxaoctan-DD8-; Sigma Chemical Co., St. Louis, Mo.).

Diluted rabbit serum samples (1:400 for gASCA and gALCA, 1:200 for aACCA) reacted for 30 minutes with specific antigens immobilised in microtitre wells. After washing away unbound serum components, antibodies specifically bound to antigen, are detected using peroxidase labeled goat anti-rabbit IgG (Zymed Laboratories Inc., San Francisco, USA) or rabbit IgA (Sigma) diluted 1:1000 and 1:50, respectively. After 30 minutes incubation, unbound conjugate is removed by washing, and chromogenic substrate (tetramethyl benzidine) is added for 15 minutes. Subsequently, a stop solution is added to terminate the enzymatic reaction. Results are expressed as optical density (OD) at 450 nm.

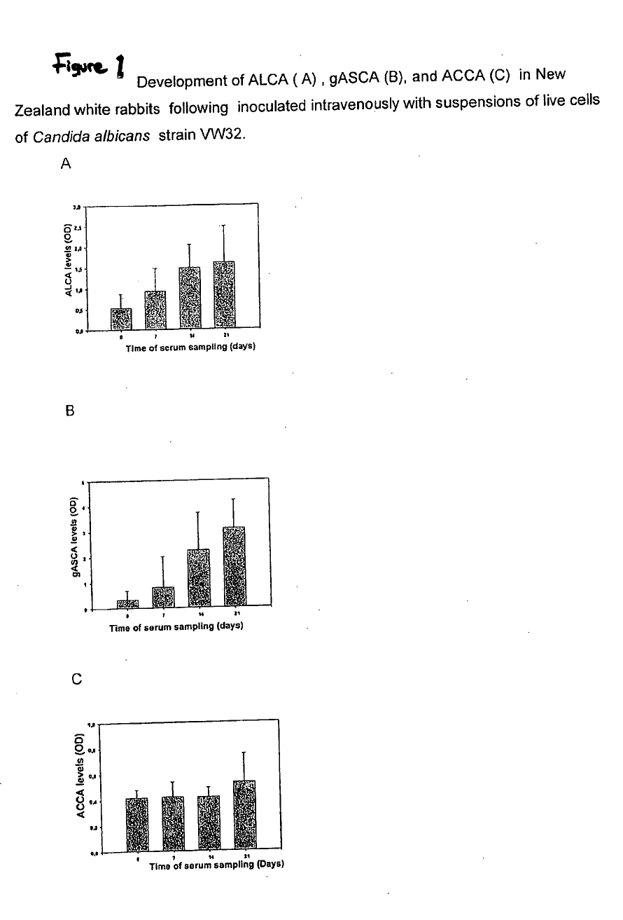

FIGS. 1A-C describe the development of OD over time from inoculation indicating the production of gASCA, ALCA and ACCA in response to the C. albicans cells inoculation.

### EXAMPLE 2

### Antibodies Against Glucan, Chitin and Saccharomyces Cerevisiae Mannan are New Biomarkers of Candida Albicans Infection that Complement Tests Based on C. Albicans Mannan

Antibodies against Saccharomyces cerevisiae mannan (ASCA) and synthetic disaccharide fragments of glucans (ALCA) and chitin (ACCA) are biomarkers of Crohn's disease (CD). Candida albicans infection generates ASCA. ALCA and ACCA as possible biomarkers of invasive C. albicans infection (ICI) was explored. ASCA, ALCA, ACCA and Candida mannan antigen and antibody detection tests were performed on 69 sera obtained sequentially from 18 patients with ICI proven by blood culture, 59 sera from CD patients, 47 sera from hospitalized and colonized (CZ) subjects, and sera from healthy controls (HC). ASCA, ALCA and ACCA levels in CD and ICI patients were significantly different from those in CZ and HC (p<0.0001). In ICI, levels increased as infection developed. Using ASCA, ALCA, ACCA and Platelia Candida tests, 100% of ICIs were detected, with the kinetics of the antibody response depending on the patient during the time-course of infection. A large number of sera presented with more than three positive tests. This is the first evidence that the detection of antibodies against chitin and glucans has diagnostic value in fungal infections and that these tests can complement other tests to achieve improved diagnosis.

Serum Samples from Patients with Invasive Candidiasis

Sixty-nine serum samples were selected retrospectively between January 2005 and December 2006 from 18 patients hospitalized in Lille University Hospital and Saint Antoine University Hospital, Paris, who developed ICI. The patients consisted of nine females and nine males (mean age: 48±19 years). The average number of sera per patient was 3.8±2.17 (Table 1). The following selection criteria were applied retrospectively: (i) fever non-responsive to antibacterial therapy but responsive to antifungal therapy; (ii) one or several positive cultures for C. albicans from blood; (iii) availability of sera within a range of 3 weeks before to 4 weeks after positive cultures; and (iv) analysis of the medical charts of patients with special attention to risk factors.

Patients with Crohn's Disease

Patients were selected retrospectively from a previous study of families recruited through the Registre des Maladies Inflammatoires Chroniques de l'Intestin du Nord-Ouest de la France (EPIMAD) and the Inflammatory Bowel Disease Registry at the University Hospital, Gasthuisberg, Leuven (Van Kruiningen HJ et al., 2007 J Clin Gastroenterol, 41:583-90). The proband was selected from each family. The diagnosis of CD was based on usual criteria and phenotypes were defined according to the Montreal classification. A total of 59 CD patients (20 males/39 females, median age 45 years, range 20-82) were selected. The age at diagnosis was known for 58 patients: <17 years for seven (12.1%), 17-40 years for 46 (79.3%), and ≧40 for five (6.6%). The disease location was known for 57 patients: 10 (17.5%) ileal (L1), nine (15%) colonic (L2), 37 (64.9%) ileo-colonic (L3), and one (1.8%) isolated upper disease (L4). Disease behavior was documented in 56 cases: 27 (48.2%) non-stricturing, non-penetrating (B1), 18 (32.1%) stricturing (B2), 11 (19.6%) penetrating (B3), and 20 (35.7%) perianal.

### Control Sera

Control sera consisted of 47 serum samples from patients (n=47) with one or two body sites (trachea, sputum, urine, stools, etc.) colonized by Candida species and hospitalized in the ICU, and 131 sera from healthy blood donors.

### Detection of Anti-C. Albicans Mannan Antibodies and Mannanemia

Antibodies to C. albicans mannan and mannanemia were detected using the Platelia® Candida Ab and Platelia Candida Ag tests (Bio-Rad Laboratories, Marnes-la-Coquette, France) as described previously (Sendid B et al., 2002 J Med Microbiol, 51:433-42).

### Detection of ASCA and Anti-Glycan Antibodies

All sera were assayed using a panel of tests that detects novel serological markers of CD (Dotan I et al., 2006 Gastroenterology, 131:366-78). This panel, named IBDX™ (Glycominds, Israel), comprises ASCA, ALCA and ACCA kits involving three antigens: S. cerevisiae mannan, laminaribioside and chitobioside, respectively. Para-nitrophenyl derivatives of laminaribioside and chitobioside and S. cerevisiae mannan were covalently bound to the surface of microtiter wells with a linker (oligomer of 1,8-diamino-3,6-dioxaoctan; Sigma Chemical Co., St. Louis, Mo.). These tests were performed according to the manufacturer's instructions. ASCA, ALCA and ACCA results were expressed as arbitrarily units (AU), which are relative to a Glycominds laboratory (gASCA, ALCA, ACCA) calibrators that are derived from a pool of patient sera with well-characterized disease. Antibody titers for each sample are calculated by dividing the average OD of the sample by the average OD of the calibrator, multiplied by the number of units denoted the calibrator tube label. The cut-off levels were set such that 97%, 100%, and 92% of the HC were below the cut-off value for ACCA, ALCA, and ASCA, respectively. The cut-off reference levels were 50 units for ASCA, 60 units for ALCA, and 80 units for ACCA.

### C. Albicans Infection in Rabbits and Follow-Up of Anti-Glycan Antibody Responses

Three New Zealand white rabbits (2-3 kg) were inoculated intravenously with 144 μL of live C. albicans VW32 yeasts (2.10⁶ yeasts/mL). Serum samples were obtained every week for 3 weeks and stored at −20° C. Antibodies against C. albicans mannan (Platelia Ab), S. cerevisiae mannan (ASCA), laminaribioside (ALCA) and chitobioside (ACCA) were detected by ELISA, which was adapted for detection of rabbit IgG with classical washing and incubation steps. Sera were diluted 1:400 for Platelia Ab, ASCA and ALCA tests and 1:200 for the ACCA test, and incubated with the corresponding antigens. Antibody binding was detected using peroxidase-labeled goat anti-rabbit IgG (Zymed Laboratories Inc., San Francisco, USA) diluted 1:1000 and tetramethyl benzidine (TMB) as a substrate (Bio-Rad, Marnes la Coquette, France).

### Monoclonal Antibody Against β-Glucans

Monoclonal antibody (mAb) 2G8, is a murine IgG2b reacting with β-glucan epitopes (WO/2006/030318). Dilutions of 2G8 (1:500-1:16000) were prepared from a concentration of 0.6 mg/mL and tested by ELISA on ALCA-microtiter plates. One-hundred microliters of each dilution was added to the wells and incubated for 1 h at 37° C. After washing in TNT (Tris 50 mM, NaCl 150 mM, HCl pH 7.5, Tween 20 0.05%), 100 μL of HRP-conjugated antibodies (goat anti-mouse IgG; Southern Biotech, USA) diluted 1:5000 in TNT were added for 1 h at 37° C. After washing in TNT, each well received 100 μL of TMB (Bio-Rad, USA). After 30 min, the reaction was stopped by adding 100 μL of blocking reagent and the plate was read at 450 nm. A mAb to Candida (EB-CA1), recognizing a mannopentaose (Jacquinot P M et al., 1998 FEMS Microbiol Lett, 169: 13 1-8) was used as a control.

### Statistical Analysis

As ASCA, ALCA and ACCA were not normally distributed, the significance of differences between two independent groups was determined by the Mann-Whitney U-test, and the significance of differences among more than two groups was determined by Kruskal-Wallis one-way analysis. In ICI patients, Spearman's rank correlation coefficients were calculated to estimate the interrelation between anti-yeast glycan antibodies or mannan levels and time. Antibody values were classified arbitrarily into four groups according to the time of serum sampling. The date of isolation of Candida species from blood culture was defined as day 0; group 1 included sera obtained during the period day-25 to day-1; group 2 (day 0 to day+15); group 3 (day+16 to day+40) and group 4 (day+41 to day+154). Statistical analysis was performed using SPSS for Windows version 11.0 (SPSS Inc.). p<0.05 was considered as statistically significant.

ASCA, ALCA, and ACCA Levels are Highly Significantly Elevated in Invasive Candidiasis and Compare with those Observed in CD Patients

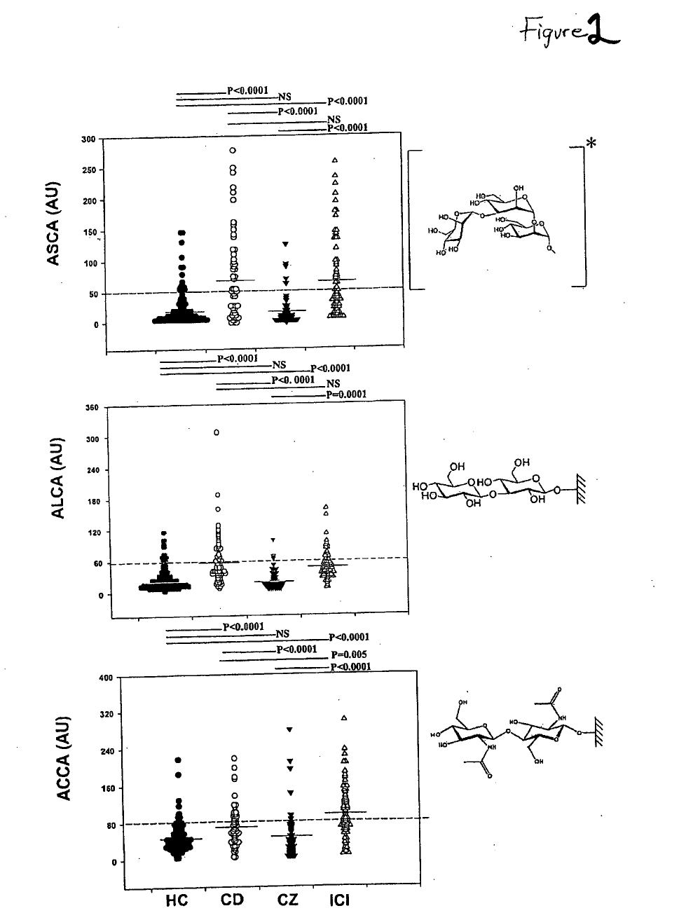

IBDX™ antibodies were detected in 69 sera from patients with ICI, 47 sera from CZ,_{—}131 sera from HC, and 59 CD sera. A significant difference in levels of ASCA, ALCA and ACCA between ICI patients and CZ/HC was observed (p<0.0001) (FIG. 2). This was similar to the difference between CD patients and HC, and between CD and CZ patients. There was no significant difference in ASCA or ALCA levels between CD and ICI patients, although ACCA levels were significantly higher in ICI (p=0.002).

ASCA, ALCA and ACCA are Generated During Experimental C. Albicans Infection and the ALCA Test Detects Antibodies Against Glucan Epitopes Protecting from C. Albicans Infection

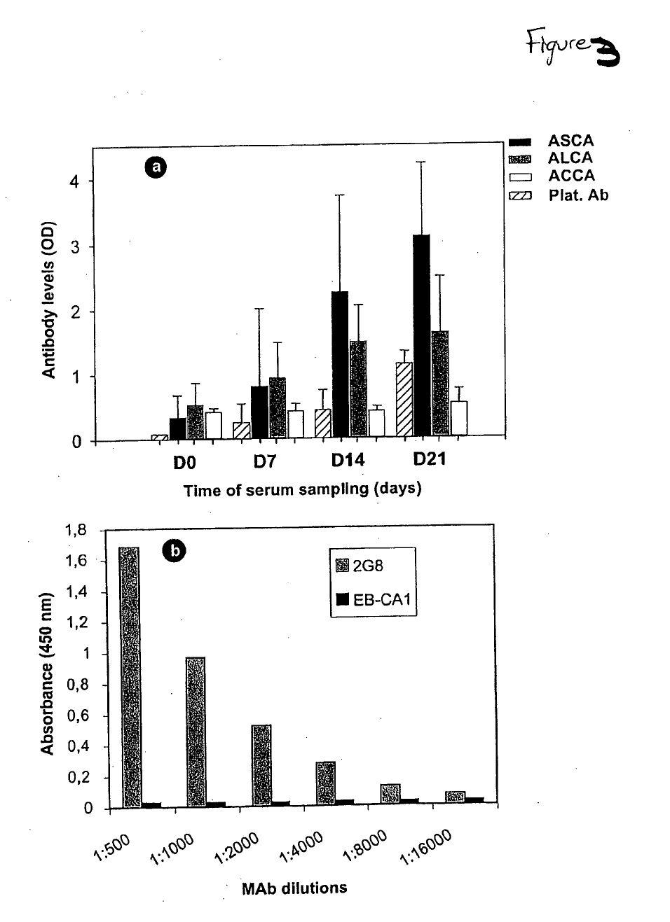

In order to assess the significance of ASCA, ALCA and ACCA in relation to C. albicans infection, rabbits were experimentally infected with C. albicans. FIG. 3 shows the results of antibody detection tests. Despite variation in background levels related to the adaptation of these tests to rabbits and differences in antibody levels between animals, ASCA and ALCA increased continuously as a result of C. albicans infection, as did anti-C. albicans mannan antibodies. ACCA levels were lower and a delayed increase could only be observed 3 weeks after infection.

It was then investigated whether ALCA could be protective antibodies as described for mAb 2G8 in experimental C. albicans infection. When different concentrations of mAb 2G8 were allowed to react with laminaribioside, a typical dose-dependent reactivity curve was observed demonstrating that laminaribioside is among the epitopes recognized by this mAb (FIG. 3b). Under the same conditions, mAb EB-CA1 against Candida mannan did not exhibit any binding activity at concentrations as high as 10 μg/mL.

### ASCA, ALCA and ACCA Increase in Individual Sera as a Result of C. Albicans Infection as do Anti-C. Albicans Mannan Antibodies

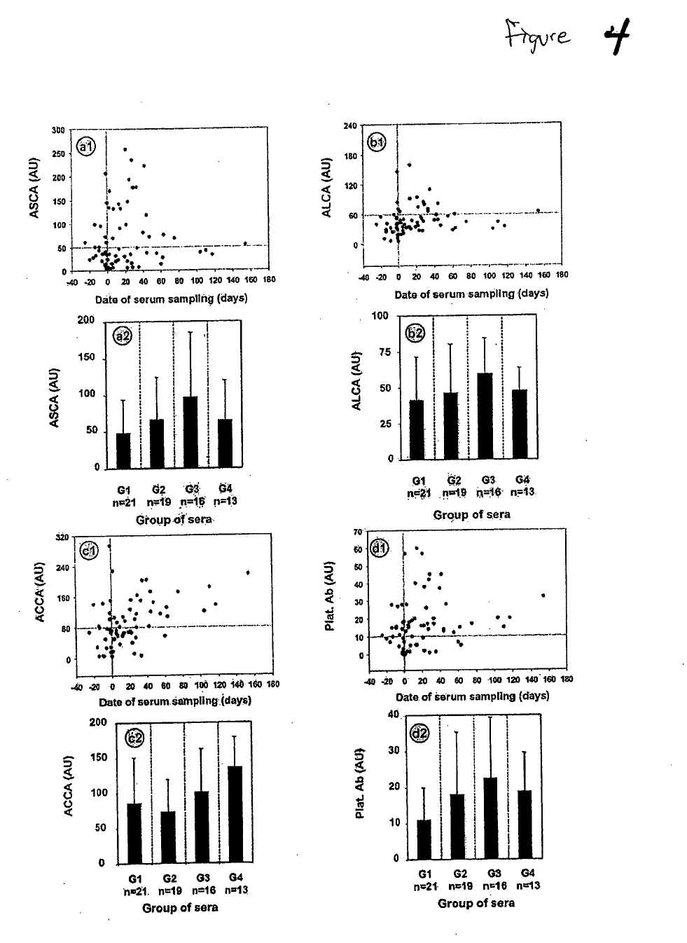

Anti-C. albicans antibody levels are known to increase during transition of C. albicans from colonization to infection, and are used as adjunct tests to blood cultures for the diagnosis of ICI. The Platelia® Ab response was compared to ASCA, ALCA and ACCA levels during systemic C. albicans infection confirmed by positive blood culture. The results are shown in FIG. 4 a1, b1, c1 and d1, respectively. The figures demonstrate that for each of the four antibodies examined, patients tested positive for the antibodies in their blood before the blood culture yielded positive results for C. albicans. The correlation between antibody levels and day when the sera were drawn was determined using Spearman's rank correlation coefficient. A correlation was observed for ACCA (p=0.0018), ALCA (p=0.019) and Platelia Ab (p=0.02), demonstrating a link between C. albicans infection and an increase in these anti-glycan antibodies.

### ASCA, ALCA, ACCA, Anti-C. Albicans Mannan Antibodies and C. Albicans Mannanemia Exhibit Different Kinetics in Individual Sera Depending on the Time of Serum Sampling

The contribution of IBDX™ and Platelia Ab and Ag tests to the diagnosis of ICI in relation to the time of serum sampling versus blood culture was investigated. After isolation of C. albicans from blood, positive antibody tests decreased: ACCA (23/48), ASCA (22/48) and ALCA (12/48), although only one of 48 sera was negative in both the IBDX™ and Platelia tests. ACCA and Platelia Ab were more frequently positive at least 1 week before the isolation of C. albicans from blood. The majority of patients presented more than three positive anti-carbohydrate antibody tests; in two of these a lower antibody response was associated with mannanemia. Table 2 shows a distribution of sera in relation to the date of isolation of C. albicans from blood (day 0) and number of positive results for each test and for a combination of tests.

The results obtained for the 18 ICI patients are summarized in Table 3, with patients listed according to the number of sera available. ASCA, ALCA and ACCA were detected in 13 (72%), 12 (66%) and 13 (72%) ICI patients, respectively, and only two patients were negative with the IBDX™ panel. For these two patients, Platelia tests were positive (twice for antigen and once for anti-mannan antibodies). Despite the limited number of sera (n=2) available from seven patients, Table 3 indicates that 78% (14/18) of patients had at least three positive tests and 67% (12/18) had at least four positive tests. Table 3 also shows that only four of the 69 sera were negative with all tests. All patients (100%) were detected if at least one positive test was considered.

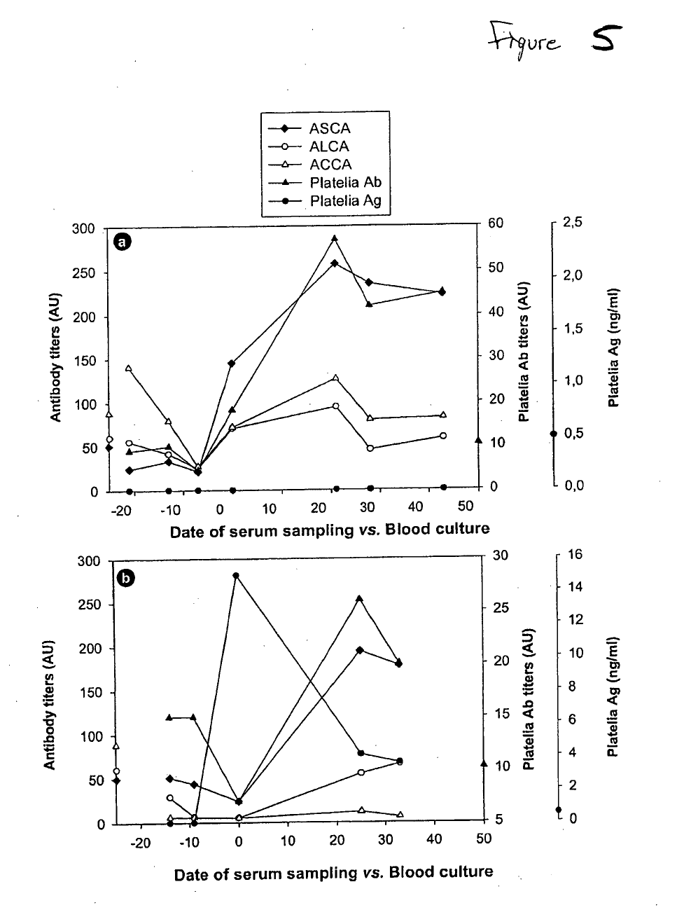

Complementation of the serological tests can also be observed when individual values are plotted in a kinetic manner during the course of C. albicans infection. FIG. 5(a, b) shows two representative examples for patients 14 and 6 (see Table 3). These two patients already had two positive tests within the 2 week period before blood cultures became positive. Both showed a sharp drop in antibodies in sera taken around the time of positive blood culture. This phenomenon is associated with a massive release of fungal molecules correlating with circulation of C. albicans in the bloodstream. This was observed for patient 6 (FIG. 4b) who had very high levels of mannan detected by the Platelia Ag test, whereas for patient 14 (FIG. 4a) the epitope detected by this test was not detected suggesting the presence of other molecules interfering with the antibody detection tests. In both patients, this period was followed by a rapid increase in all anti-carbohydrate antibodies although the antibody levels to a given antigen differed between patients. Nevertheless, at least three different tests were simultaneously positive for both patients during this period.

Low levels of ACCA were detected in a high percentage of the control population. In yeasts, which are present in the diet and human gut, chitin provides cross-linking and strength to the cell wall polysaccharide scaffolding. Increased chitin synthesis is a response to cell wall weakening. In C. albicans, the cell wall of the invasive hyphal form contains three times more chitin than the yeast form. In relation to the presence of low levels of ACCA in the control population, the data demonstrates that two pathogenic situations result in an increase in ACCA, namely CD and infection by C. albicans. In this latter pathology, kinetic analysis of antibody levels during the time-course of the disease clearly demonstrated a cause-to-effect relationship between C. albicans infection and increase in ACCA levels before the blood culture yielded positive results for C. albicans. In addition, the anti-chitobioside antibody response is maintained at high levels many weeks after the candidemic episode.

The data indicate that ALCA are generated in patients during C. albicans infection, and in rabbits with experimental ICI. C. albicans

cell wall glucans are linked to proteins (Klis F M et al., 2002 FEMS Microbiol Rev, 26:239-56) and are therefore able to induce an antibody response (Breinig F K et al., 2004 Microbiology, 150:3209-18; Ecker M R et al., 2006 J Biol Chem, 281:11523-9). Prior to the invention described herein, it has not been possible to detect human antibodies against glucans due to the lack of a reproducible test. The ALCA test provides a positive response to this obstacle.

TABLE 1
Clinical features of patients with systemic C. albicans infection

| | | | | Date of serum sampling in | |
|---|---|---|---|---|---|
| Patient n° | Sex, Age | Hospital Ward | No. of sera | relation to blood culture (days) | Candida species |
| 1 | M, 43 | ICU* | 7 | (40; 47; 75; 104; 110; 117; 154) | C. albicans |
| 2 | F, 71 | Oncology | 2 | (−1; 63) | C. albicans |
| 3 | M, 57 | ICU | 2 | (7; 14) | C. albicans |
| | | | | | C. glabrata |
| 4 | M, 18 | ICU | 4 | (−14; −7; 3; 23) | C. albicans |
| 5 | F, 73 | ICU | 3 | (−5; −2; 9) | C. albicans |
| 6 | F, 21 | Hematology | 5 | (−14; −9; 0; 25; 33) | C. albicans |
| 7 | M, 51 | Surgery | 9 | (−2; 5; 12; 19; 26; 33; 44; 55; 62) | C. albicans |
| 8 | M, 31 | Hematology | 5 | (−2; 5; 22; 27; 60) | C. albicans |
| 9 | F, 75 | Surgery | 2 | (−1; 2) | C. albicans |
| 10 | F, 49 | ICU | 2 | (−2; 6) | C. albicans |
| 11 | M, 68 | Nephrology | 2 | (−1; 1) | C. albicans |
| 12 | F, 28 | Surgery | 2 | (−1; 13) | C. albicans |
| 13 | M, 15 | ICU | 2 | (−1; 13) | C. albicans |
| 14 | M, 42 | ICU | 7 | (−20; -13; -6; 0; 21; 28; 42) | C. albicans |
| 15 | F, 80 | ICU | 3 | (−25; −16; −9) | C. albicans |
| 16 | F, 53 | Neurosurgery | 3 | (−2; 21; 44) | C. albicans |
| 17 | M, 49 | Surgery | 3 | (9; 15; 29) | C. albicans |
| 18 | F; 45 | ICU | 6 | (−1; 3; 6; 22; 28; 35) | C. albicans |

*ICU: Intensive care unit

TABLE 2
Distribution of sera in relation to the date of isolation of C. albicans from blood (day 0)
and number of positive results for each test and for a combination of tests.

| | | | | | | | | | Number of sera negative for all |
|---|---|---|---|---|---|---|---|---|---|
| | | | | | | | | Negative | tests (N), or positive for one (1+), |
| Time of serum | Number | | | | Platelia | Platelia | Positive | Platelia | two to three (2-3+) and four to |
| collection | of sera | ASCA | ALCA | ACCA | Ab | Ag | Platelia tests | tests | five (4-5+) tests |
| >1 week before | 8 | 2 | 0 | 4 | 5 | 1 | 6 | 2 | N: 0 |
| | | | | | | | (75%) | | 1+: 3 |
| | | | | | | | | | 2-3+: 5 |
| 1 week before | 13 | 4 | 2 | 7 | 5 | 5 | 9 | 4 | N: 3 |
| | | | | | | | (69%) | | 1+: 2 |
| | | | | | | | | | 2-3+: 7 |
| 0-7 days after | 12 | 5 | 2 | 3 | 7 | 9 | 12 | 0 | 0: 0 |
| | | | | | | | (100) | | 1+: 3 |
| | | | | | | | | | 2-3+: 8 |
| | | | | | | | | | 4+: 1 |
| >7 days after | 36 | 17 | 10 | 20 | 28 | 9 | 30 | 6 | N: 1 |
| | | | | | | | (83%) | | 1+: 5 |
| | | | | | | | | | 2-3+: 22 |
| | | | | | | | | | 4-5+: 8 |

TABLE 3
Results of mannanemia, antimannan antibodies and IBDX tests in patients with invasive Candida infection

| | No. of | | | | | | No. of sera | No. of tests for which |
|---|---|---|---|---|---|---|---|---|
| | available | ASCA | ALCA | ACCA | | | negative for | patient was positive |
| Patient N^{o} | sera | (50 AU) | (60 AU) | (80 AU) | Platelia Ab | Platelia Ag | all tests | at least once |
| 7 | 9 | 0/9 | 2 | 6 | 8/9 | 1/9 | 0 | 4/5 |
| 1 | 7 | 4 | 1 | 7 | 7 | 3 | 0 | 5/5 |
| 14 | 7 | 4/7 | 2/7 | 2 | 5/7 | 0/7 | 1 | 4/5 |
| 18 | 6 | 0 | 3/6 | 6/6 | 0 | 4/6 | 0 | 4/5 |
| 6 | 5 | 3/5 | 1/5 | 0 | 4/5 | 3/5 | 0 | 4/5 |
| 8 | 5 | 0 | 0 | 0 | 3/5 | 2/5 | 0 | 2/5 |
| 4 | 4 | 4/4 | 0 | 1 | 4/4 | 2/4 | 0 | 4/5 |
| 17 | 3 | 2 | 1 | 3 | 3 | 0 | 0 | 4/5 |
| 15 | 3 | 2/3 | 0/3 | 1/3 | 2/3 | 1/3 | 0 | 4/5 |
| 5 | 3 | 0/3 | 1/3 | 0/3 | 3/3 | 3/3 | 0 | 3/5 |
| 16 | 3 | 2/3 | 1/3 | 2/3 | 2/3 | 0/3 | 0 | 4/5 |
| 10 | 2 | 2/2 | 0 | 1 | 2/2 | 1/2 | 0 | 4/5 |
| 2 | 2 | 1/2 | 0/2 | 1/2 | 0/2 | 0/2 | 1 | 2/5 |
| 9 | 2 | 2/2 | 2/2 | 2/2 | ½ | 1/2 | 0 | 5/5 |
| 11 | 2 | 0 | 0 | 0 | 0 | 1/2 | 1 | 1/5 |
| 12 | 2 | 1 | 1 | 0 | ½ | 0/2 | 1 | 3/5 |
| 13 | 2 | 1 | 2 | 2 | 1 | 0 | 0 | 4/5 |
| 3 | 2 | 2 | 0 | 0 | 0 | 2 | 0 | 2/5 |

What is claimed is:

1. A method of diagnosing an invasive or systemic Candida albicans infection or a risk of developing said infection, the method comprising:
providing a blood sample from a subject;
detecting a level of an anti-chitobioside antibody (ACCA) in said sample by binding to a carbohydrate reagent comprising an isolated chitobioside molecule;
detecting a level of an anti-laminaribioside antibody (ALCA) in said sample by binding to a carbohydrate reagent comprising an isolated laminaribioside molecule; and
diagnosing a Candida albicans infection by detection of an elevated level of said antibodies in said blood sample relative to a reference level.

2. The method of claim 1, wherein said method comprises
detecting ACCA and ALCA in said test sample, and diagnosing a fungal infection in said subject by detection of an elevated level of said antibodies in said blood sample relative to a reference level.

3. The method of claim 1, wherein said method further comprises
detecting gASCA in said test sample, and diagnosing a fungal infection in said subject by detection of an elevated level of said antibodies in said blood sample relative to a reference level.

4. The method of claim 2, wherein said method further comprises
detecting gASCA in said test sample, and diagnosing a fungal infection in said subject by detection of an elevated level of said antibodies in said blood sample relative to a reference level.

5. The method of claim 1, further comprising comparing amounts of mannans in said test sample to the amounts of mannans in a control sample or reference level from a control population or reference sample of one or more individuals that do not have a fungal infection, wherein a fungal infection is diagnosed if the manann amount in said test sample is higher then the amount of in control population or reference sample.

6. The method of claim 1, further comprising comparing amounts of β1,3 glucans in said test sample to the amounts of β1,3 glucans in a control sample or reference level from a control population of one or more individuals that do not have a fungal infection, wherein a fungal infection is diagnosed if the β1,3 glucans amount in said test sample is higher then the amount of β1,3 glucans in control population.

7. The method of claim 1, further comprising comparing amount of chitin in said test sample to the amounts of chitin in a control sample or reference level from a control population of one or more individuals that do not have a fungal infection, wherein a fungal infection is diagnosed in said subject if the chitin amount in said test sample is higher than mannan amount in control population.

8. The method of claim 1, further comprising comparing amounts of anti-mannan antibodies in said test sample to the amounts of anti-mannan antibodies in a control sample or reference level from a control population of one or more individuals that do not have a fungal infection.

9. The method of claim 1, wherein said subject is characterized as immunocompromised or comprising one or more criteria for ICU admission.

10. The method of claim 1, wherein said sample is taken from said subject before said subject is positive in a blood culture test for said fungal infection.

11. The method of claim 1, wherein said fungal infection is caused by a Candida spp.

12. The method of claim 1, wherein said Candida spp. is Candida albicans.

14. The method of claim 1, wherein said fungal infection is systemic candidiasis, invasive aspergillosis, allergic bronchopulmonary aspergillosis, or pneumocystosis.

15. The method of claim 1, wherein said amounts of said antibody are determined by measuring binding of said antibody to a synthetic glycan.

16. The method of claim 1, wherein said amounts of said antibody are determined by measuring binding of said antibody to a polysaccharide containing the glycan antigen.

17. The method of claim 1, wherein said test sample is a biological fluid.

18. The method of claim 1, wherein biological fluid is whole blood, serum, plasma, spinal cord fluid, urine, tears or saliva.

19. The method of claim 1, wherein said biological fluid is serum.

20. A method of diagnosing an invasive fungal infection in a subject, the method comprising:
comparing amounts of ACCA in a test sample from said subject to amounts of said antibody in a control sample or reference level from a control population of one or more individuals that do not have a fungal infection; and diagnosing a fungal infection in said subject by detection of an elevated level of said antibodies in said blood sample relative to a reference level.

21. A method of diagnosing an invasive fungal infection in a subject, the method comprising:
comparing amounts of ALCA in a test sample from said subject to amounts of said antibody in a control sample or reference level from a control population of one or more individuals that do not have a fungal infection; and diagnosing a fungal infection in said subject by detection of an elevated level of said antibodies in said blood sample relative to a reference level.
